(19) United States
(12) Patent Application Publication
(10) Pub. No.: US 2019/0096514 A1
(43) Pub. Date: Mar. 28, 2019

(54) PREVENTIVE CARE ALERT SYSTEM

(71) Applicant: Curae Systems, Inc., San Diego, CA (US)

(72) Inventors: Robert Nascenzi, San Diego, CA (US); Christopher Fletcher, San Diego, CA (US)

(21) Appl. No.: 16/131,918

(22) Filed: Sep. 14, 2018

Related U.S. Application Data
(60) Provisional application No. 62/562,239, filed on Sep. 22, 2017.

(51) Int. Cl. G16H 20/00; G16H 50/20

(52) U.S. Cl. CPC G16H 20/00; G16H 50/20

(57) ABSTRACT

Systems, devices, and methods for providing client care alert are disclosed herein. The method can include storing symptom information related to a plurality of medical conditions. The method can include receiving client information via a first application and generating a care plan for the client based on the client information and the symptom information. The method can include generating a set of tasks to be completed during a care session based on the care plan. The method can include receiving task input related to completion of the set of tasks. The task input may indicate the presence of at least one symptom related to a medical condition. Thereafter, the method can include transmitting a message including an identification of the at least one symptom to a second application.

### CROSS REFERENCE TO RELATED APPLICATIONS

This application claims benefit to U.S. Provisional Application No. 62/562,239, filed Sep. 22, 2017, entitled, “MEDICAL CARE ALERT SYSTEM,” the contents of which are hereby incorporated by reference in their entirety.

### BACKGROUND

### Technical Field

This application related to a medical care alert system. More particularly, this application relates to a system for ensuring that a client of non-clinical, in-home care receives appropriate and timely medical attention.

### Related Art

Some care plans produced by non-clinical home care agencies are not modified based on conditions discovered during the assessment process, unless the home care agency is manually instructed to do so by the client or family member. In addition, even if the care plan is modified to account for the discovered conditions, symptoms related to the condition are not easily accessible and sometimes are not even available for review by a caregiver or a concerned third party. Thus, there is no satisfactory solution for ensuring that a client receives timely medical attention from a medical professional for all conditions as warranted by observed symptoms.

### SUMMARY

This disclosure provides systems, methods, and computer-readable media for facilitating appropriate and timely medical attention for a client.

One aspect of the disclosure provides a method for use in an automated system for improved client care. The method can include storing symptom information associated with symptoms related to a plurality of medical conditions in a memory. The method can include receiving client information associated with a client by one or more processors via a first application, the one or more processors being communicatively coupled to the memory. The method can include generating, by the one or more processors, a care plan for the client, the care plan being based on the client information and the symptom information and including a set of tasks to be completed during a care session. The method can include. receiving, by the one or more processors, task input related to completion of one or more tasks of the set of tasks by a caregiver, the task input indicating presence of at least one symptom related to a medical condition of the plurality of medical conditions. The method can include transmitting a message including an identification of the at least one symptom to a second application based on the task input.

Another aspect of the disclosure provides a device for providing a client care alert. The device can have a memory operable to store symptom information associated with symptoms related to a plurality of medical conditions. The device can have one or more processors communicatively coupled to the memory. The one or more processors can receive client information associated with a client by one or more processors via a first application, the one or more processors being communicatively coupled to the memory. The one or more processors can generate a care plan for the client based on the client information and the symptom information. The one or more processors can generate a set of tasks to be completed by a caregiver during a care session based on the care plan. The one or more processors can receive task input related to completion of one or more tasks of the set of tasks, the task input indicating the presence of at least one symptom related to a medical condition of the plurality of medical conditions. The one or more processors can transmit a message including an identification of the at least one symptom to a second application based on the task input.

Another aspect of the disclosure provides a method for providing a care alert. The method can include receiving, by at least one hardware processor of a platform, client information for a client via at least one network from a client application of a first user. The method can include generating, by the at least one hardware processor, a care plan for the client based on the received client information. The method can include retrieving one or more symptoms for a medical condition from at least one database based on the client information. The method can include adding the retrieved one or more symptoms to the care plan. The method can include generating a set of one or more tasks based on the care plan. The method can include transmitting the set of one or more symptoms over the at least one network to a client application of a second user when the tasks identifies that one or more of the symptoms are present. When the tasks identify one or more symptoms, at least one of the one or more symptoms the method can include prompting the second user to contact a medical professional regarding at least one of the one or more symptoms.

Additional details and advantages provided by the disclosure will become apparent with a review of the following description.

### BRIEF DESCRIPTION OF THE DRAWINGS

The details of embodiments of the present disclosure, both as to their structure and operation, can be gleaned in part by study of the accompanying drawings, in which like reference numerals refer to like parts, and in which:

FIG. 1 is a functional block diagram of an embodiment of a communication system for in-home care plan management;

FIG. 2 is a functional block diagram of an embodiment of a processing system for in-home care plan management for use with the system of FIG. 1; and

FIG. 3 is a flowchart of an embodiment of a method for managing client in-home care plans;

### DETAILED DESCRIPTION

This disclosure presents descriptions of systems, methods, and non-transitory computer-readable media for managing in-home client care plans and facilitating appropriate and timely medical attention for a client. Additionally, embodiments of the described methods and systems can be employed in other care arrangements (e.g., nursing homes).

After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

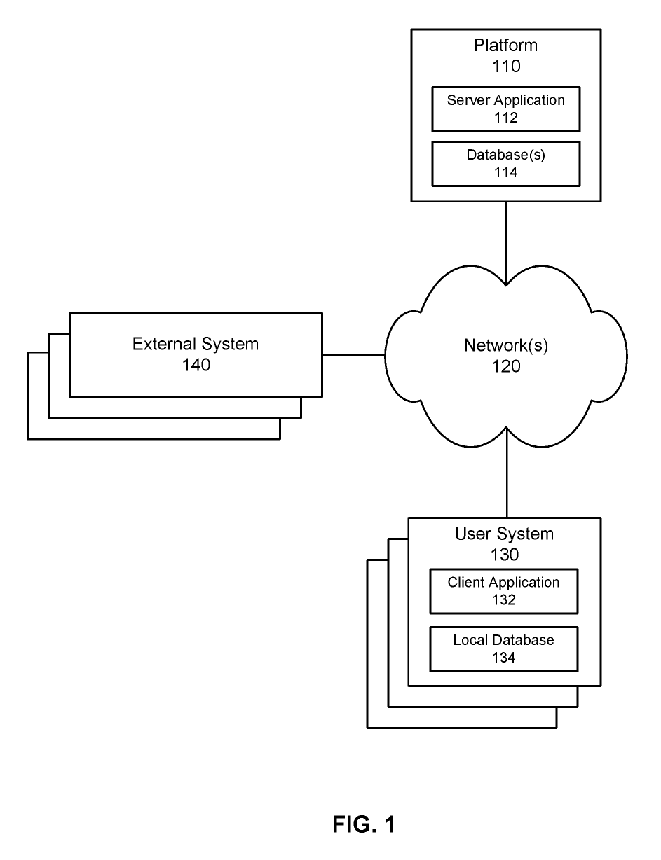

FIG. 1 is a functional block diagram of an embodiment of a communication system for in-home care plan management. A communication system 100 can have a platform 110 that can host and/or execute one or more of the various functions, processes, methods, and/or software modules described herein. The platform 110 can have one or more servers and associated processors for the performance of the various functions, processes, methods, and/or software modules described herein. The platform 110 can have or be communicatively connected to a server application 112 and/or one or more databases 114. The databases 114 can have one or more memories for storing data and instructions for the performance of tasks.

The platform 110 can also be communicatively connected to one or more user systems 130 via one or more networks 120. The networks 120 can include a local area network (LAN) or wide area network (WAN) as required. The platform 110 may also be communicatively connected to one or more external systems 140 (e.g., websites, apps, other platforms, etc.) via the one or more networks 120. The network(s) 120 may comprise the Internet, and the platform 110 may communicate with user system(s) 130 through the Internet using standard transmission protocols, such as HyperText Transfer Protocol (HTTP), Secure HTTP (HTTPS), File Transfer Protocol (FTP), FTP Secure (FTPS), SSH FTP (SFTP), and the like, as well as proprietary protocols. In an embodiment, the platform 110 may not comprise dedicated servers, but may instead comprise cloud instances, which utilize shared resources of one or more servers. It should also be understood that the platform 110 can have collocated servers or cloud instances (e.g. virtual machines). Furthermore, while the platform 110 is illustrated as being connected to various systems through a single set of network(s) 120, it should be understood that the platform 110 may be connected to the various systems via different sets of one or more networks. For example, the platform 110 may be connected to a subset of user systems 130 and/or external systems 140 via the Internet, but may be connected to one or more other user systems 130 and/or external systems 140 via an intranet. It should also be understood that user system(s) 130 may be any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, Automated Teller Machines, and the like. In addition, while only a few user systems 130 and external systems 140, one server application 112, and one set of database(s) 114 are illustrated, it should be understood that the infrastructure may comprise any number of user systems, server applications, and databases.

The platform 110 may comprise servers which host one or more websites and/or web services. In embodiments in which a website is provided, the website may comprise one or more user interfaces, including, for example, webpages generated in HyperText Markup Language (HTML) or other language. The platform 110 can transmit or serve these user interfaces in response to requests from user system(s) 130. In some embodiments, these user interfaces may be served in the form of a wizard, in which case two or more user interfaces may be served in a sequential manner, and one or more of the sequential user interfaces may depend on an interaction of the user or user system with one or more preceding user interfaces. The requests to the platform 110 and the responses from the platform 110, including the user interfaces, may both be communicated through the network(s) 120, which may include the Internet and/or a wireless network, using standard communication protocols (e.g., HTTP, HTTPS). These user interfaces or web pages may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to the platform 110. The platform 110 may also respond to other requests from the user system(s) 130.

The platform 110 may further have, be communicatively coupled with, or otherwise have access to the one or more database(s) 114. For example, the platform 110 may have one or more database servers which manage the one or more databases 114. The user system 130 or server application 112 executing on platform 110 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Sybase™, Access™, and the like, including cloud-based database instances and proprietary databases. Data may be sent to the platform 110, for instance, a variety of protocols such as HTTP, FTP, etc. This data, as well as other requests, may be handled, for example, by server-side web technologies, such as Django™, ASP.NET, Ruby on Rails™, NodeJS™, or other software module (e.g., application 112), executed by platform 110.

In embodiments in which a web service is provided, the platform 110 may receive requests from external system(s) 140, and provide responses in eXtensible Markup Language (XML), JavaScript Object Notation (JSON), and/or any other suitable or desired format. In such embodiments, the platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, etc., described herein. For example, in such an embodiment, a client application 132 executing on one or more user system(s) 130 may interact with a server application 112 executing on the platform 110 to execute one or more or a portion of one or more of the various functions, processes, methods, and/or software modules described herein. Client application 132 may be “thin,” in which case processing is primarily carried out server-side by server application 112 on the platform 110. A basic example of a thin client application is a browser application, which simply requests, receives, and renders webpages at user system(s) 130, while the server application on the platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on the platform 110, at any point along this spectrum between “thin” and “thick,” depending on the design goals of the particular implementation. In any case, the application described herein, which may wholly reside on either the platform 110 (e.g., in which case, the server application 112 performs all processing) or user system(s) 130 (e.g., in which case, the client application 132 performs all processing) or be distributed between the platform 110 and user system(s) 130 (e.g., in which case, the server application 112 and client application 132 both perform processing), can comprise one or more executable software modules that implement one or more of the processes, methods, or functions of the application(s) described herein.

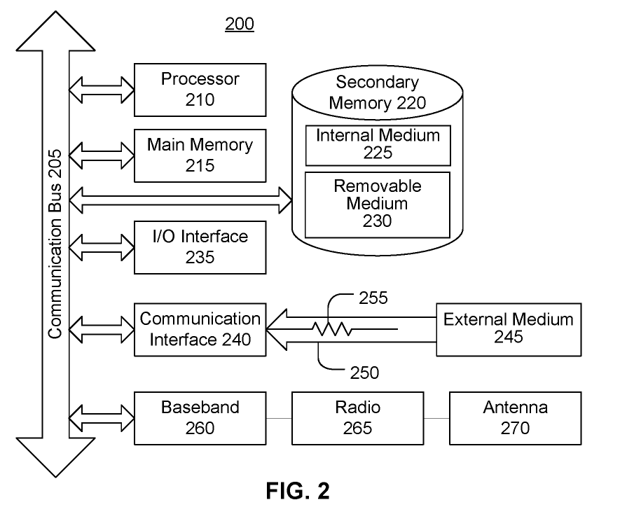

FIG. 2 is a functional block diagram of an embodiment of a processing system for in-home care plan management for use with the system of FIG. 1. For example, a system 200 may be used as or in conjunction with one or more of the mechanisms, processes, methods, or functions (e.g., to store and/or execute the application or one or more software modules of the application) described herein. In addition, the system 200 may represent components of the user system(s) 130. Additionally, the platform 110 and the external system(s) 140, and/or other processing devices described herein can be implemented using similar or the same system 200. The system 200 can be a server or any conventional personal computer, or any other processor-enabled device that is capable of wired or wireless data communication. Other computer systems and/or architectures may be also used, as will be clear to those skilled in the art.

The system 200 preferably includes one or more processors, such as processor 210. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 210.

The processor 210 can be coupled to a communication bus 205. The communication bus 205 may include a data channel for facilitating information transfer between storage and other peripheral components of the system 200. Furthermore, the communication bus 205 may provide a set of signals used for communication with the processor 210, including a data bus, address bus, and control bus (not shown). The communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, or standards promulgated by the Institute of Electrical and Electronics Engineers (IEEE) including IEEE 488 general-purpose interface bus (GPIB), IEEE 696/S-100, and the like.

The system 200 can include a main memory 215 and may also include a secondary memory 220. The main memory 215 provides storage of instructions and data for programs executing on the processor 210, such as one or more of the functions and/or modules discussed above. It should be understood that programs stored in the memory and executed by the processor 210 may be written and/or compiled according to any suitable language, including without limitation C/C++, Java, JavaScript, Perl, Visual Basic, .NET, and the like. The main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The secondary memory 220 may optionally include an internal memory 225 and/or a removable medium 230. The removable medium 230 is read from and/or written to in any well-known manner. The removable storage medium 230 may be, for example, a magnetic tape drive, a compact disc (CD) drive, a digital versatile disc (DVD) drive, other optical drive, a flash memory drive, etc.

The removable storage medium 230 is a non-transitory computer-readable medium having stored thereon computer-executable code (e.g., disclosed software modules) and/or data. The computer software or data stored on removable storage medium 230 is read into the system 200 for execution by processor 210.

In alternative embodiments, the secondary memory 220 may include other similar means for allowing computer programs or other data or instructions to be loaded into the system 200. Such means may include, for example, an external storage medium 245 and a communication interface 240, which allows software and data to be transferred from the external storage medium 245 to the system 200. Examples of the external storage medium 245 may include an external hard disk drive, an external optical drive, an external magneto-optical drive, etc. Other examples of the secondary memory 220 may include semiconductor-based memory such as programmable read-only memory (PROM), erasable programmable read-only memory (EPROM), electrically erasable read-only memory (EEPROM), or flash memory (block-oriented memory similar to EEPROM).

As mentioned above, the system 200 may include a communication interface 240. The communication interface 240 allows software and data to be transferred between the system 200 and external devices (e.g. printers), networks, or other information sources. For example, computer software or executable code may be transferred to the system 200 from a network server via the communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, Bluetooth™, a network interface card (NIC), a wireless data card, a communications port, an infrared interface, an IEEE 1394 fire-wire, or any other device capable of interfacing the system 200 with a network or another computing device. The communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via the communication interface 240 are generally in the form of electrical communication signals 255. These signals 255 may be provided to the communication interface 240 via a communication channel 250. In an embodiment, the communication channel 250 may be a wired or wireless network, or any variety of other communication links. The communication channel 250 carries the signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (i.e., computer programs, such as the disclosed application, or software modules) is stored in the main memory 215 and/or the secondary memory 220. Computer programs can also be received via the communication interface 240 and stored in the main memory 215 and/or the secondary memory 220. Such computer programs, when executed, enable the system 200 to perform the various functions of the disclosed embodiments as described elsewhere herein.

In this description, the term “computer-readable medium” is used to refer to any non-transitory computer-readable storage media used to provide computer-executable code (e.g., software and computer programs) to the system 200. Examples of such media include the main memory 215, the secondary memory 220 (including the internal memory 225, the removable medium 230, and the external storage medium 245), and any peripheral device communicatively coupled with communication interface 240 (including a network information server or other network device). These non-transitory computer-readable mediums are means for providing executable code, programming instructions, and software to the system 200.

In an embodiment that is implemented using software, the software may be stored on a computer-readable medium and loaded into the system 200 by way of the removable medium 230, the I/O interface 235, or the communication interface 240. In such an embodiment, the software is loaded into the system 200 in the form of electrical communication signals 255. The software, when executed by the processor 210, preferably causes the processor 210 to perform the features and functions described elsewhere herein.

In an embodiment, the I/O interface 235 provides an interface between one or more components of the system 200 and one or more input and/or output devices. Example input devices include, without limitation, keyboards, touch screens or other touch-sensitive devices, biometric sensing devices, computer mice, trackballs, pen-based pointing devices, and the like. Examples of output devices include, without limitation, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and the like. The I/O interface 235 can include one or more user interfaces for execution of the methods and processes described herein.

The system 200 may also include optional wireless communication components that facilitate wireless communication over a voice network and/or a data network. The wireless communication components comprise an antenna system 270, a radio system 265, and a baseband system 260. In the system 200, radio frequency (RF) signals are transmitted and received over the air by the antenna system 270 under the management of the radio system 265.

In one embodiment, the antenna system 270 may comprise one or more antennae and one or more multiplexors (not shown) that perform a switching function to provide the antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, the radio system 265 may comprise one or more radios that are configured to communicate over various frequencies. In an embodiment, the radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from the radio system 265 to the baseband system 260.

If the received signal contains audio information, then the baseband system 260 decodes the signal and converts it to an analog signal. Then the signal is amplified and sent to a speaker. The baseband system 260 also receives analog audio signals from a microphone. These analog audio signals are converted to digital signals and encoded by the baseband system 260. The baseband system 260 also codes the digital signals for transmission and generates a baseband transmit audio signal that is routed to the modulator portion of the radio system 265. The modulator mixes the baseband transmit audio signal with an RF carrier signal generating an RF transmit signal that is routed to the antenna system 270 and may pass through a power amplifier (not shown). The power amplifier amplifies the RF transmit signal and routes it to antenna system 270, where the signal is switched to the antenna port for transmission.

The baseband system 260 is also communicatively coupled with processor 210, which may be a central processing unit (CPU). The processor 210 has access to the data storage areas 215 and 220. The processor 210 is preferably configured to execute instructions (i.e., computer programs, such as the disclosed application, or software modules) that can be stored in the main memory 215 or the secondary memory 220. Computer programs can also be received from the baseband processor 260 and stored in the main memory 215 or in the secondary memory 220, or executed upon receipt. Such computer programs, when executed, enable the system 200 to perform the various functions of the disclosed embodiments. For example, the data storage areas 215 or 220 may include various software modules.

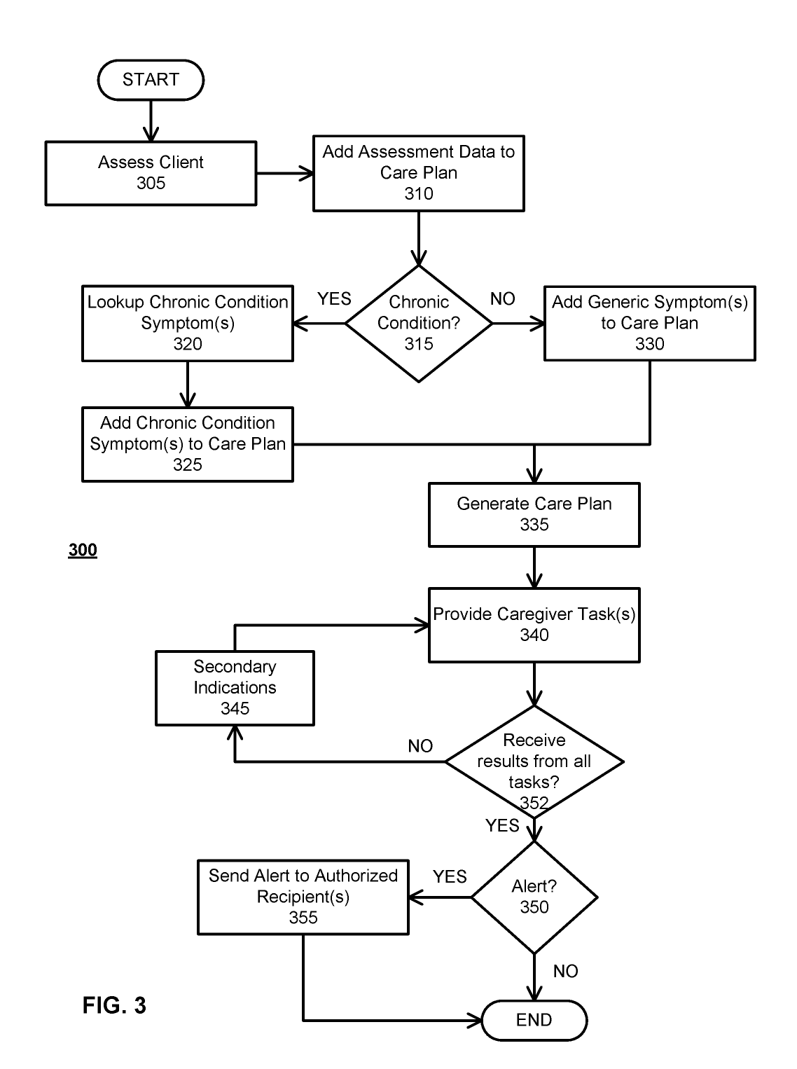

FIG. 3 is a flowchart of an embodiment of a method for managing client in-home care plans. A method 300 can allow prevention of a client medical emergency. While the method 300 is illustrated with a certain arrangement of steps, the method 300 may be implemented with fewer, more, or different steps and a different arrangement or ordering of steps.

It should be understood that the processes described in connection with the method 300 may be embodied in one or more software modules that are executed by one or more hardware processors, e.g., as the application discussed above (e.g., server application 112, client application 132, and/or a distributed application comprising both server application 112 and client application 132), which may be executed wholly by processor(s) 210 of the platform 110, wholly by processor(s) 210 of the user system(s) 130, or may be distributed across the platform 110 and the user system(s) 130 such that some portions or modules of the application are executed by the platform 110 and other portions or modules of the application are executed by the user system(s) 130 and respective components of the system 200. The described process may be implemented as instructions represented in source code, object code, and/or machine code. These instructions may be executed directly by the hardware processor(s), or alternatively, may be executed by a virtual machine operating between the object code and the hardware processors. In addition, the disclosed application may be built upon or interfaced with one or more existing systems.

While not specifically stated in the description of every step of the process 300, the client, the caregiver, or a third party can interact with the various applications (e.g., the server application 112 and the client application 132) via a user interface (e.g., the I/O interface 235). In addition, the server application 112 and the client application 132 can perform tasks via the processor 210 and the memory 215, for example. Accordingly, one or more of the various steps of the process 300 can be performed by one or more instantiations of the system 200 (FIG. 2) in coordination with one or more components of the system 100 (FIG. 1).

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for ease of description. Specific functions or steps can be moved from one component, block, module, circuit, or step to another without departing from the invention.

As illustrated, process 300 includes a client assessment shown in block 305. For instance, when a home care agency begins service for a new client, a home care consultant may assess the client to create a care plan. In addition, the assessment may be repeated periodically and upon changes in the condition of the client. In an embodiment, this assessment includes the collection of basic client information, such as demographics of the client (e.g., name, address, gender, age, etc.), medical, mental, and/or behavioral conditions of the client, allergies of the client, medications and/or supplements being used by the client, ambulation of the client, and/or the like. In some embodiments certain photos can be included in the assessment and added to the client information. For example, baseline photos of various physical symptoms of the client (e.g., an ankle before swelling or after swelling, etc.) can be included in the assessment or client information. The client information can be stored in one or more profiles within a memory or database (e.g., the database 114, the database 134, or the memory 215). Additionally, the collected information can be updated periodically, at the request of the client or an authorized person. In addition, when assessing the medical condition of a client, the home care consultant may discover that the client has one or more chronic or acute medical conditions.

In block 310, the assessment information, collected in block 305, may be input in the application. For example, the assessment information, including the client information and any discovered chronic or acute conditions, may be entered by the home care consultant, client, and/or other individual via a user interface (e.g., the I/O interface 235) of the client application 132. The input assessment information, including any conditions discovered during the assessment, can be saved to the memory 215 and added to (e.g., recorded and listed in) the care plan for the client. Thus, the client application 132 can receive input including the assessment information. Known conditions or known symptoms can also be added to the profile or client information and saved to memory.

In block 315, the application (e.g., server application 112) can determine whether any chronic medical conditions (e.g., congestive heart failure, COPD, kidney disease, prior heart attack, etc.) or acute or common medical conditions (e.g., urinary tract infection, dehydration, pneumonia, etc.) were added to the care plan in block 310. If chronic or acute condition(s) were added to the care plan in block 310 (i.e., “YES” in block 315), the process 300 proceeds to block 320. Otherwise, if no chronic or acute conditions were added to the care plan in block 310 (i.e., “NO” in block 315), process 300 proceeds to block 330. In block 320, the application (e.g., server application 112) automatically looks up (e.g., in database 114) each chronic or acute condition, added to the care plan in block 310, to identify a set of one or more symptoms associated with each chronic or acute condition.

In block 325, each set of symptom(s), returned during the look-up process in block 320 for a chronic or acute condition in the care plan, can be added to the care plan. In an embodiment, adding a symptom to the care plan comprises adding a task for checking the symptom to the care plan. For example, if a symptom involves swelling or other changes to some body part, a task of checking that body part for swelling or other changes may be added to the care plan. Similarly, if a symptom involves increases or decreases in weight, a task of weighing a client may be added to the care plan. The application may look up symptoms within the database 114 (e.g., the memory 215) to retrieve a task associated with the symptom. In certain cases, a plurality of symptoms, retrieved in block 320, may map to the same task, in which case the application may only add a single instance of that task to the care plan in block 325, instead of adding a plurality of the same, redundant task.

In block 330, the application may automatically add a generic set of one or more symptoms to the care plan. For example, if no chronic or acute conditions are identified during the assessment in block 305, the application can automatically include generic or common symptoms (e.g., confusion, shortness of breath, incontinence, chest tightness, weight gain/loss, etc.) in the care plan. In an embodiment, this may include adding a set of one or more tasks for checking the generic or common set of symptom(s). The generic set of symptom(s) may include symptoms of common conditions or a comprehensive set of potential conditions. In this manner, the tasks added to the care plan in block 330 can be used to identify conditions in a client, even if those conditions have not been previously identified as being applicable to the client. In an alternative embodiment, block 330 can be performed even when the application determines in block 315 that one or more chronic or acute medical conditions have been added to the care plan. In either case, the inclusion of generic symptoms can be used to identify and alert authorized parties when one or more symptoms persist over time or arise suddenly. Alternatively, the block 330 step can be performed for all clients including those with chronic conditions.

In block 335, the application (e.g., server application 112 or the processor 210) can generate a care plan to be used by a caregiver. The care plan may define one or more care giving shifts (e.g., daily shifts, weekly shifts, shifts for specific dates and/or times, etc.) and tasks to be performed during each care giving shift.

In block 340, the processor 210 or the server application 112 can generate a set of tasks for guiding the caregiver through an episode of care giving from the care plan. The client application 132 can then present or otherwise provide the tasks to the caregiver (e.g., in the user interface 235 of the client application 132). Block 340 may be initiated when a caregiver begins a care session or a shift of care giving (e.g., “punches in”) via one or more inputs in a user interface of a client application 132 of the caregiver's user system 130. “Punching in” for a shift may require the caregiver to log in (e.g., provide credentials such as a username and password) to a caregiver account of the application, and select one or more inputs for initiating the caregiver's shift. Similarly, ending, or “punching out” of a shift may require the caregiver to select one or more inputs, in the user interface of the client application 132 of the caregiver's user system 130, for ending the caregiver's shift.

In an embodiment of block 340, the server application 112 may generate a set of tasks for a given time period of care giving (e.g., the current day) and provide the set of tasks to the client application 132 being executed on the caregiver's user system 130 (e.g., in response to the caregiver checking in for his or her shift). The caregiver can interact with the client application 132, for example, via the I/O interface 235. The client application 132 can then prompt the caregiver to perform each task, throughout the shift, in a user interface (e.g., the user interface 235) of client application 132. The tasks may be presented in a particular sequence (e.g., to maximize efficiency in the caregiver's time) and/or particular tasks may be presented at certain times during the time period of care giving (e.g., client application 132 may prompt the caregiver to provide medication to the client after each meal period).

In some embodiments, tasks may include the addition of a required input (e.g., a yes or no) related to specific symptoms determined during client care. Some examples are:

For diabetes:
Did they seem excessively thirsty or hungry?
Calculate weight change, if any.
Did they have to urinate more often than usual?

For COPD (chronic obstructive pulmonary disease):
Did they cough frequently?
If yes, was mucus coughed up (e.g., a productive cough)?
Did they experience more shortness of breath than usual?

For Kidney Disease:
Did they complain that their food tasted like metal?
Does their face look puffy or hands look swollen?
Were they itchy or unusually cold?

The caregiver may have to perform specific actions (e.g., weigh the client or interact with the client) and provide task input or input specific data (e.g., a weight) in order to answer the specific questions and satisfy the required tasks. In some embodiments, certain tasks may further require the caregiver to compare current state or condition of the client with a previously recorded or baseline condition, previous photo, or historical description of the client or associated ailments or conditions.

At decision block 352, the processor 210, for example, can determine whether the results of each task (e.g., an input to the user interface or the processor 210 of the client application 132) are received from the caregiver. For example, as the caregiver completes each task provided in the user interface of the client application 132, the caregiver may select an input that indicates that the task has been completed, and/or, in cases in which it is appropriate, provide other input related to the task (e.g., an indication of whether or not a body part is swollen or inflamed for a task that requires the caregiver to check for swelling of that body part, the weight of the client for a task that requires the caregiver to weigh the client, etc.). It should be understood that the inputs may be in the form of check boxes, radio buttons, text boxes, drop-down menus, etc. It should also be understood that actions associated with block 352 may entail receiving multiple task results at different times throughout the time period of care giving.

At decision block 352, if not all of the tasks from block 340 have been completed, one or more secondary indications may be presented to the caregiver at block 345. In some embodiments, such secondary indications may be a flashing screen, flashing text, a noise, or other indication set as a reminder to the caregiver to complete the set of tasks provided. The method 300 can then return to block 340. In an embodiment, if one or more of the tasks presented to the caregiver are not completed at decision block 352, the client application 132 may prohibit the caregiver from “punching out” of or otherwise ending a care session until all tasks provided in block 340 have been completed. Once all of the tasks have been completed at decision block 342, the method 300 can proceed to decision block 350.

At decision block 350, the application (e.g., the processor 210) can determine if an alert should be issued. Such an alert can be a client care alert based on the answers to the questions, or the measurements/statistics provided in response to the set of tasks. For example, server application 112 may receive the task results input by the caregiver in block 345. The server application 112 may receive these task results in real time as they are received at the caregiver's client application 132, periodically throughout the caregiver's shift, at the end of the caregiver's shift, or at any other frequency. As the server application 112 receives the task results from the caregiver's client application 132, server application 112 may apply one or more rules to the task results to identify symptoms from the task results, and attempt to match any identified symptoms to one or more diseases or other conditions. If symptoms are identified and those symptoms match one or more conditions (e.g., at or above a predetermined confidence threshold) that require medical attention and/or certain symptoms persist for a predetermined period of time (e.g., indicating a chronic condition), server application 112 determines that an alert should be generated (i.e., “YES” in block 350), and proceeds to block 355. Otherwise, if no symptoms are identified or the identified symptoms do not match any conditions that require medical attention and/or are not persistent, no alert is generated (e.g., “NO” in block 350), and process 300 ends. The caregiver may end a care session following the completion of the method 300. In some embodiments, after decision block 350, the method 300 can return to block 340 if further tasks remain incomplete or for a subsequent care session provided by the caregiver. In other embodiments, if another care session is begun with an existing client, then the method 300 may pick up at block 335. For example, an existing client may not require an additional assessment (e.g., block 310) because the client's conditions are already known.

At block 355, one or more alerts can be generated and sent to one or more authorized recipients. The alerts may identify the condition(s) requiring medical attention or otherwise indicate that the client should seek medical attention, and may be sent via any communication means, including, without limitation, email message, Short Message Service (SMS) message or other text message protocol, Multimedia Messaging Service (MMS) message, a telephone call with an audio recording, and/or the like.

The authorized recipients (e.g., family members, guardians, etc.) may be set and identified by the application via account settings of the client. The information associated with the authorized recipients can be stored by the server application 112 (e.g., in the memory 215). For example, each client managed by the application can be associated with an account and each recipient can be authorized via registration with the client's account by providing contact information for the recipient while logged in to the client's account. Consequently, the application (e.g., the server application 112 or the client application 132) can automatically retrieve one or more of the authorized (third party) recipients' contact information from the client's account settings and transmit notifications to the authorized recipients when necessary.

Thus, in an embodiment, the disclosed application not only automates the modification of the care plan to account for newly discovered conditions (e.g., chronic or acute diseases discovered during a client assessment), but it also automates the sending of critical information to concerned individuals (e.g., family members or other loved ones) regarding other common medical conditions (urinary tract infection, de-hydration, pneumonia, etc.). By associating specific medical conditions with their symptoms, the application can generate alerts and send those alerts to concerned individuals (e.g., loved ones' mobile devices), to notify the concerned individuals regarding the client's condition and possible need for medical attention, prior to the condition requiring emergency medical attention.

In some embodiments, the database 114 can store information associated with a plurality of chronic medical conditions and their respective or at least their most common symptoms. As an example, suppose that the home care consultant discovers, during the assessment, that a new client has a history of congestive heart failure (e.g., based on the client's response to a question). In this case, the home care consultant or caregiver may select an input, associated with congestive heart failure (e.g., a checkbox next to a label of “Congestive Heart Failure”), within an assessment user interface of client application 132 (e.g., in block 310). In response to the selection of the input for congestive heart failure, the application may perform a lookup on congestive heart failure (e.g., client application 132 may identify congestive heart failure in a message to server application 112, and server application 112 may then perform a lookup in database 114) to identify a set of symptoms associated with congestive heart failure, such as swollen ankles and sudden weight gain. Based on this lookup, the application may automatically insert a set of tasks corresponding to the set of symptoms into the client's care plan. Consequently, a caregiver may be instructed (e.g., via the set of caregiver tasks in block 340) to check the client's ankles for swelling and weigh the client at approximately the same time each day. If the caregiver fails to complete either of these tasks, the caregiver may be prevented from “punching out” of or otherwise ending his or her shift at block 345 until the tasks are completed.

Continuing the example, the application (e.g., the server application 112) automatically stores (e.g., in the database 114) and monitors the information collected by the caregiver as he or she completes the tasks (e.g., decision block 342). If the application receives an indication that the client's ankles are swollen (e.g., the caregiver has specified that the client's ankles are swollen during completion of the task of checking the client's ankles) and/or that the client has gained a certain threshold amount of weight or has gained weight at or above a certain threshold rate of weight gain, the application may automatically generate and send an alert (e.g., email, SMS or MMS message, automated telephone call with audio recording, in-app alert, etc.) to one or more authorized third parties (e.g., doctor, family members, such as spouse, children, grandchildren, etc.). The alert notifies each recipient of the client's condition (e.g., providing an indication of the symptoms and/or the condition(s) of which the symptoms are indicative). The recipient(s) can then ensure that the client receives appropriate and timely medical attention for the condition (e.g., by scheduling an appointment for the client to see a doctor or otherwise prompting the client to see a medical professional, etc.). Therefore, the application increases the likelihood that the client will see a medical professional before the client's condition worsens (e.g., to a point that the client requires treatment in an emergency room).

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The accompanying claims and their equivalents are intended to cover such forms or modifications as would fall within the scope of the disclosure. The various components illustrated in the figures may be implemented as, for example, but not limited to, software and/or firmware on a processor or dedicated hardware. Also, the features and attributes of the specific example embodiments disclosed above may be combined in different ways to form additional embodiments, all of which fall within the scope of the disclosure.

In one or more exemplary embodiments, the functions described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in processor-executable instructions that may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include random access memory (RAM), read-only memory (ROM), electrically erasable programmable read-only memory (EEPROM), FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

The foregoing method descriptions and the process flow diagrams are provided merely as illustrative examples and are not intended to require or imply that the operations of the various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an,” or “the” is not to be construed as limiting the element to the singular.

The various illustrative logical blocks, modules, and algorithm operations described in connection with the embodiments disclosed herein may be implemented as electronic hardware, computer software, or combinations of both. To clearly illustrate this interchangeability of hardware and software, various illustrative components, blocks, modules, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present inventive concept.

It is understood that the specific order or hierarchy of blocks in the processes/flowcharts disclosed is an illustration of exemplary approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not meant to be limited to the specific order or hierarchy presented.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

Although the present disclosure provides certain example embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

What is claimed is:

1. A method for use in an automated system for improved client care comprising:
storing symptom information associated with symptoms related to a plurality of medical conditions in a memory;
receiving client information associated with a client by one or more processors via a first application, the one or more processors being communicatively coupled to the memory;
generating, by the one or more processors, a care plan for the client, the care plan being based on the client information and the symptom information and including a set of tasks to be completed during a care session;
receiving, by the one or more processors, task input related to completion of one or more tasks of the set of tasks by a caregiver, the task input indicating a presence of at least one symptom related to a medical condition of the plurality of medical conditions; and
transmitting a message including an identification of the at least one symptom to a second application based on the task input.

2. The method of claim 1 further comprising:
determining that fewer than all tasks of the set of tasks have been completed; and
providing a secondary indication via the first application to the caregiver to complete the set of tasks.

3. The method of claim 2, wherein the secondary indications comprise one of an alert provided via the first application and preventing the caregiver from ending the care session.

4. The method of claim 1, wherein the message includes a prompt to contact a medical professional regarding the presence of at least one symptom.

5. The method of claim 1 wherein the set of tasks further comprises checking a state of the at least one symptom related to a medical condition.

6. The method of claim 1 further comprising:
identifying the medical condition of the plurality of medical conditions based on the client information after receiving the client information;
retrieving an identification of symptoms related to the medical condition from the memory, and
adding the symptoms to the care plan before generating the set of tasks.

7. The method of claim 1, further comprising adding a set of generic symptoms to the care plan.

8. The method of claim 1, wherein the first application is different from the second application.

9. The method of claim 8, wherein the first application is associated with a caregiver, and wherein the second application is associated with a concerned third party.

10. A non-transitory computer-readable medium having instructions stored therein, wherein the instructions, when executed by the one or more processors, cause the one or more processors to perform the method of claim 1.

11. A device for providing a client care alert comprising:
a memory operable to store symptom information associated with symptoms related to a plurality of medical conditions;
one or more processors communicatively coupled to the memory and operable to
receive client information associated with a client by one or more processors via a first application, the one or more processors being communicatively coupled to the memory;
generate a care plan for the client based on the client information and the symptom information;
generate a set of tasks to be completed by a caregiver during a care session based on the care plan;
receive task input related to completion of one or more tasks of the set of tasks, the task input indicating a presence of at least one symptom related to a medical condition of the plurality of medical conditions; and
transmitting a message including an identification of the at least one symptom to a second application based on the task input.

12. The device of claim 11 wherein the one or more processors is further operable to:
determine that fewer than all tasks of the set of tasks have been completed; and
provide a secondary indication via the first application to the caregiver to complete the set of tasks.

13. The device of claim 12, wherein the secondary indications comprise one of an alert provided via the first application and preventing the caregiver from ending the care session.

14. The device of claim 11, wherein the message includes a prompt to contact a medical professional regarding the presence of at least one symptom.

15. The device of claim 11 wherein the one or more processors is further operable to:
identify the medical condition of the plurality of medical conditions based on the client information after receiving the client information;
retrieve an identification of symptoms related to the medical condition from the memory, and
add the symptoms to the care plan before generating the set of tasks.

16. A method for providing a care alert comprising:
receiving, by at least one hardware processor of a platform, client information for a client via at least one network from a client application of a first user;
generating, by the at least one hardware processor, a care plan for the client based on the received client information;
retrieving one or more symptoms for a medical condition from at least one database based on the client information; and
adding the retrieved one or more symptoms to the care plan;
generating a set of one or more tasks based on the care plan; and
when the tasks identifies that one or more of the symptoms are present, transmitting the set of one or more symptoms over the at least one network to a client application of a second user,
wherein, when the tasks identify one or more symptoms, at least one of the one or more symptoms, prompting the second user to contact a medical professional regarding at least one of the one or more symptoms.

17. The method of claim 16, wherein the first user and the second user are different.

18. The method of claim 17, wherein the first user is a caregiver, and wherein the second user is a concerned third party.

19. The method of claim 16 further comprising adding, by the at least one hardware processor, one or more generic symptoms to the care plan when the client information does not identify any conditions.

20. The method of claim 16 further comprising:
receiving, at the at least one hardware processor, a result of the one or more tasks via the at least one network from the client application of the second user;
identifying one or more symptoms from the result;
matching the identified one or more symptoms to at least one condition; and
transmitting an alert over the at least one network to one or more recipients, wherein the alert specifies the matched at least one condition.
